(19) United States
(12) Plant Patent Application Publication
(10) Pub. No.: US 2002/0108158 P1
(43) Pub. Date: Aug. 8, 2002

(54) Geranium plant named 'Tikorg'

(75) Inventor: Wilhelm Elsner, Dresden (DE)

Correspondence Address: Russell D. Orkin, 700 Koppers Building, 436 Seventh Avenue, Pittsburgh, PA, 15219-1818, US

(73) Assignee: Eslner pac Jungpflanzen

(21) Appl. No.: 09/753,976

(22) Filed: Jan. 3, 2001

(51) Int. Cl. A01H 5/00

(52) U.S. Cl. PLT/330

(57) ABSTRACT

A new and distinct zonal geranium plant with bright red colored flowers above medium green foliage with distinct zonation. The plant may be grown in a pillar form when supported.

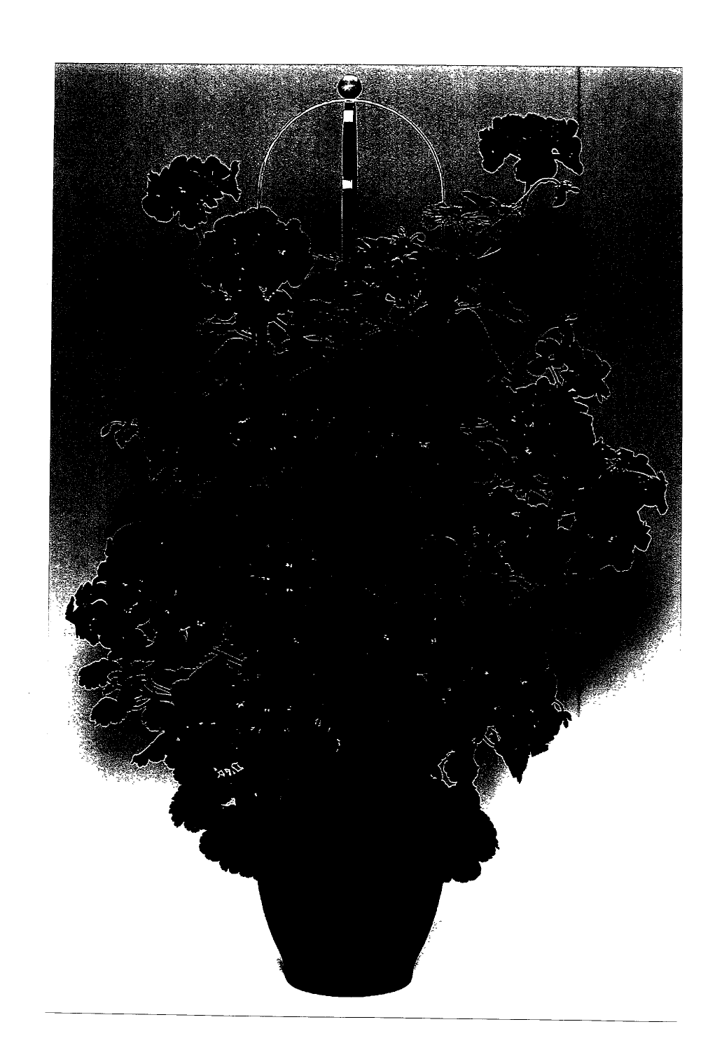

### BACKGROUND OF THE INVENTION

The present invention comprises a new and distinct cultivar of Pelargonium x hortorum known by the varietal names 'pac® Antik Orange, Tikorg^{(s)} and pac® Pillar Orange, Tikorg^{(s)} (Breeder No. P-6137). The new variety was discovered in 1996 in a selected breeding program in Dresden, Germany. The new variety is a selection from a population of seedlings resulting from breeding and crossing of diploid group 95K-43. The new variety exhibits vigorous growth and has the long internodes and strong anthocyanin markings of its heritage but with more intense flower color, larger flowers and suitability for ground cover. The new variety was first asexually reproduced by cuttings in Dresden, Germany. The new variety has been trial and field tested at Connellsville and has been found to retain its distinctive characteristics and remain true to type through successive propagations.

The cultivar, when grown in a glass greenhouse in Connellsville, Pennsylvania using natural light and temperatures of 60° F. night and 68° F. day, has a response time of six weeks from a rooted cutting to flowering in a 2 inch pot and a response time of 10-12 weeks to a saleable pillar product. The response time was determined on plants grown in soilless media employing constant fertilizer 200-250 parts per million of nitrogen and potassium in full light.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Connellsville, Pa. The color readings were taken indoors under 200-220 foot candles of fluorescent cool white light. Color references are primarily to The R.H.S. Colour Chart of The Royal Horticultural Society of London.

Plant:

Classification.—Botanical: Pelargonium x hortorum. Commercial: Zonal geranium.

Form.—Tall bush.

Height from media surface.—24-36 inches.

Growth.—Free branching, lateral to upright growth.

Strength.—If grown in pillar form, support required.

Foliage.—Stalked leaf attachment.

Rooting time.—14-21 days depending on conditions.

Leaves.—Size: Length: 5.0-5.5 cm. Width: 6.5-7.2 cm. Shape: Reniform; cordate base. Margin: Crenate. Texture: Pubescent. Color: Upper surface: Green Group 137A. Lower surface: Green Group 138B. Zone: Greyed-Purple Group 187A. Ribs and veins: Venation: Palmate. Color: Yellow-Green Group 144B.

Petioles.—Color: Yellow-Green Group 144A. Length: 6.5-7.0 cm.

Stem.—Color: Green Group 137C. Internode Length: 2.5-4.5 cm.

The bud:

Shape when tightly closed, no color showing.—Overall: Semi-spherical. Individual bud: Elongated elliptical (oval).

Size when tightly closed, no color showing.—Overall: 2.5 cm across. Individual bud: 0.5-0.6 cm wide; 0.7-0.8 cm long.

Number of buds per cluster.—10-12.

Inflorescence:

Blooming habit.—Continuous; flowering. Size: 8.0-10.0 cm across. Borne: Flower on umbel; umbel on pedicel; pedicel on peduncle.

Flowers.—Form: Open flat to slightly cupped. Size: Diameter: 4.3-4.8 cm. Depth: 0.5-1.0 cm. Petals: Color: Upper surface: Red Groups 41C and 43C. The upper two petals have darker areas of Red Group 41B and sometimes Red Group 41A with dark veining of Red Group 42A. The lower petals have less significant veining of Red Group 41B. Lower surface: Red Groups 41C and 41D and Red Group 38A; some with darker veining showing through and darker areas of Red Group 41A. Shape: Round at top drawn out to a narrower base. Size: Length: 2.0-2.5 cm. Width: 1.8-2.4 cm. Margin: Complete. Number per flower: 5.

Texture and appearance.—Smooth, reflective.

Tonality from a distance.—Bright orange-coral color with deeper values of orange and highly reflective.

Petaloids.—None.

Pedicel.—Length: 3.2-3.7 cm. Color: Greyed-Red Group 178A with Yellow-Green Group 144D.

Peduncle.—Length: 14-15 cm. Color: Yellow-Green Group 144A.

Disease/pest resistance.—No unusual susceptibility to diseases or pests has been noted to date.

Reproductive organs:

Stamens.—Anthers: 0.2 cm. Filaments: Length: 0.4-0.6 cm. Color: White with peach-coral tips. Pollen: Orange.

Pistils.—Number: One. Length: 0.7-0.8 cm. Stigma: Number: One. Color: Orange. Style: Length: 0.3 cm. Color: Orange.

Ovaries.—Length: 0.5 cm. Width: 0.2-0.3 cm. Color: Green.

Fruit.—None observed.

### GENERAL CHARACTERISTICS

1. ‘Tikorg’ is unique to the geranium industry.

2. ‘Tikorg’ is a very vigorous grower which allows it to be trained as a pillar.

3. ‘Tikorg’ is very floriferous.

I claim:

1. A new and distinct variety of geranium plant substantially as shown and described.
